(19) United States
(12) Patent Application Publication
(10) Pub. No.: US 2016/0062211 A1
(43) Pub. Date: Mar. 3, 2016

(54) NON-LINEAR OPTICAL MATERIAL AND NON-LINEAR OPTICAL ELEMENT USING SAME

(71) Applicant: FUJIFILM Corporation, Tokyo (JP)

(72) Inventors: Akihiro KANEKO, Ashigarakami-gun (JP); Kimiatsu NOMURA, Ashigarakami-gun (JP); Masataka SATO, Ashigarakami-gun (JP); Masaomi KIMURA, Ashigarakami-gun (JP)

(73) Assignee: FUJIFILM CORPORATION, Tokyo (JP)

(21) Appl. No.: 14/934,687

(22) Filed: Nov. 6, 2015

Related U.S. Application Data
(63) Continuation of application No. PCT/JP2014/061784, filed on Apr. 25, 2014.

(30) Foreign Application Priority Data
May 9, 2013 (JP) 2013-099567

(51) Int. Cl. G02F 1/361; C07D 207/44

(52) U.S. Cl. CPC G02F 1/3617; C07D 207/44; G02F 1/3612

(57) ABSTRACT

There is provided an organic non-linear optical material containing a compound represented by the Formula (I), and the Formula (I) is defined as herein, and a polymer binder:

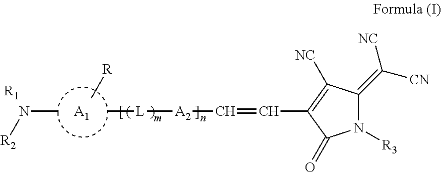

and an optical element comprising the organic non-linear optical material, and an optical modulator comprising the organic non-linear optical material, and a compound represented by the Formula (I) and the formula (I) is defined as herein.

### CROSS REFERENCE TO RELATED APPLICATION

This is a continuation of International Application No. PCT/JP2014/061784 filed on Apr. 25, 2014, and claims priority from Japanese Patent Application No. 2013-099567 filed on May 9, 2013, the entire disclosures of which are incorporated herein by reference.

### BACKGROUND OF THE INVENTION

1. Field of the Invention

The present invention relates to an organic non-linear optical material which is preferably used for a non-linear optical element, the non-linear optical element being useful in the fields of optoelectronics and photonics to which an optical modulator, an optical switch, an optical integrated circuit, an optical computer, an optical memory, a wavelength conversion element, a hologram element, and the like, which are useful in the fields using light of optical information communication, optical information processing, imaging, and the like, can be applied.

2. Description of the Related Art

Along with the development of the information society, a number of attempts to use optical techniques for transmission, processing, and recording of information have been made. Under such circumstances, a material (non-linear optical material) exhibiting a non-linear optical effect has attracted attention in the fields of optoelectronics and photonics. The non-linear optical effect is a phenomenon in which, when a strong electric field (optical electric field) is applied to a material, a non-linear relationship is established between the generated electric polarization and the applied electric field. The non-linear optical material refers to a material which significantly exhibits such non-linearity. As a non-linear optical material using a secondary non-linear response, a material generating a second harmonic or a material exhibiting a Pockels effect (linear electro-optic effect) which causes a change in refractive index in linear proportion to an electric field is known. In particular, the application of the latter material to an electro-optic (EO) modulator or a photorefractive element has been considered. Further, it is expected that the latter material will exhibit piezoelectric and pyroelectric properties, and the application of the latter material to various fields is expected.

As a secondary non-linear optical material, hitherto, an inorganic non-linear optical material such as lithium niobate or potassium dihydrogen phosphate has been put into practice and widely used. However, recently, an organic material has attracted attention due to the following reasons: 1) high non-linearity; 2) high response speed; 3) high optical damage threshold; 4) high applicability to various molecular designs; and 5) superior manufacturing aptitude. Therefore, the implementation of the organic material has been actively studied and researched.

However, in order to exhibit a secondary non-linear optical effect, it is necessary that polarization induced by an electric field lacks the center of inversion symmetry, and it is necessary that a molecule exhibiting a non-linear optical effect or a non-linear optical response group is arranged in a structure lacking the center of inversion symmetry of the material. Therefore, the organic non-linear optical material is roughly divided into two types including: a material in which the organic compound having non-linear optical activity is crystallized in a crystal structure having no center of symmetry (hereinafter, referred to as “crystalline organic non-linear optical material”); and a material in which the organic compound having non-linear optical activity is dispersed in or bonded to a polymer binder so as to be oriented using arbitrary means (hereinafter, referred to as “polymer-based organic non-linear optical material”).

In the crystalline organic non-linear optical material, it is known that extremely high non-linear optical performance can be exhibited. However, since it is difficult to manufacture large organic crystals required for manufacturing an element, the strength of the organic crystals is significantly low, and thus there is a problem such as damages in an element manufacturing step. On the other hand, in the polymer-based organic non-linear optical material, due to the polymer binder, favorable characteristics such as film formability and mechanical strength which are useful for manufacturing an element are obtained, and the potential for implementation is high. Therefore, the polymer-based organic non-linear optical material is promising.

In the related art using the polymer-based organic non-linear optical material, in order to arrange a molecule exhibiting a non-linear optical effect or a non-linear optical response group in a structure lacking the center of inversion symmetry, a configuration of introducing a molecule exhibiting a non-linear optical effect or a non-linear optical response group into the polymer binder so as to orient a dipole using, for example, an electric field has been widely used. The orientation control using an electric field is called “poling”, and a poled organic polymer is called “electric field-oriented polymer (poled polymer)”. That is, there is a method including: applying a high voltage to a polymer-based organic non-linear optical material at a temperature of a glass transition point or higher of a base polymer so as to orient a molecule exhibiting a secondary non-linear optical effect or a dipole as a response group; and cooling the polymer-based organic non-linear optical material to freeze the orientation of the dipole using an electric field. For example, an electro-optic (EO) modulator which is manufactured using the above method is known.

In addition, it is known that an organic compound having a high electron-attracting or high electron-donating group or an organic compound having a long π conjugated bond group has high non-linear optical characteristics. For example, an organic compound having a tricyanopyrroline skeleton as a high electron-attracting group or an organic compound having a tricyanopyrroline skeleton and a long π conjugated bond group has been reported (for example, U.S. Pat. No. 7,307,173B).

On the other hand, JP1987-216794A (JP-S62-216794A) describes a cyanomethylene oxopyrroline-based pigment, and JP 1993-072670A (JP-H5-072670A) describes a pyrroline-based dye compound having a cyanomethylene group which is used as a silver halide photographic material.

### SUMMARY OF THE INVENTION

However, a molecule exhibiting a secondary non-linear optical effect or a dipole of a response group which is oriented by poling undergoes thermal orientation relaxation over time. Accordingly, there is a problem in that non-linear optical characteristics of the material deteriorate.

Therefore, in the polymer-based organic non-linear optical material, an organic compound having high non-linear optical activity and a polymer binder having high film formability, mechanical strength, and the like and capable of stably maintaining the orientation state of the organic compound having high non-linear optical activity are required.

In order to realize a small-sized and low-driving-voltage EO modulator, it is necessary to introduce a high concentration of an organic compound having high non-linear optical activity into a polymer binder and to orient the organic compound with a high degree of order.

However, the organic compound in which the non-linear optical characteristics are improved using the above-described method essentially has a rod-shaped structure having a high dipole moment. This structure tends to be highly crystalline and has a problem in that the compatibility with a polymer binder is poor. The compound described in U.S. Pat. No. 7,307,173B is highly crystalline and has poor compatibility with a polymer binder. Therefore, when the compound is mixed with a polymer binder to form a film, bleed-out may occur over time.

An object of the present invention is to solve the above-described problems of the related art.

That is, an object of the present invention is to provide: an organic non-linear optical material in which not only non-linear optical performance but also compatibility with a polymer binder are improved by using a specific organic compound having non-linear optical activity which is superior in non-linear optical performance and the like; and a non-linear optical element including the organic non-linear optical material.

As a result of repeating synthesis and evaluation to solve the above-described problems, the present inventors found that, by using compounds represented the following Formulae (I) to (III) which contain a substituted amino group as an electron-donating group, a tricyanopyrroline skeleton as an electron-attracting group, and a π conjugated chain having a specific substituent, high non-linear optical activity and superior compatibility with a polymer binder can be simultaneously realized.

Further, surprisingly, the compounds represented by the following Formulae (I) to (III) are also superior in orientation during electric field poling. A significant increase not only in compatibility with a polymer but also in orientation during electric field poling, which is obtained by the compounds according to the present invention, is inconceivable in the related art. The compounds also have high compatibility with a polymer binder having a high glass transition temperature. Even when the compounds are dispersed in or bonded to a polymer binder, deterioration such as bleed-out does not occur. Therefore, superior non-linear optical characteristics can be stably maintained for a long period of time. Accordingly, the present inventors found that the organic non-linear optical material according to the present invention can solve the above-described problems, thereby completing the present invention.

[1] An organic non-linear optical material including a compound represented by the following Formula (I) and a polymer binder:

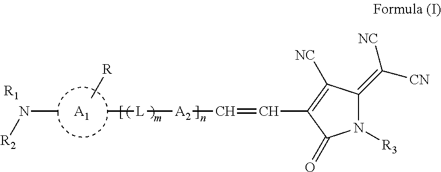

(wherein R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₁ and A₂ each independently represents an aromatic group; L represents —CR₆═CR₇—, —C≡C—, —N═CR₈—, or —CR₉═N— (R₆, R₇, R₈, and R₉ each independently represents a hydrogen atom, a substituted or unsubstituted alkyl group, or a substituted or unsubstituted aryl group); m represents an integer of 0 or 1; n represents an integer of 0 to 2; plural L's, A₂'s and m's may be the same as or different from each other; R has 3 to 30 carbon atoms and is represented by the following Formula (II); and R may be singular or plural, and plural R's may be the same as or different from each other):

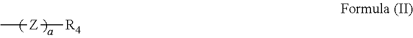

(wherein Z represents —O—, —S—, —CO—, —SO—, —SO₂—, or —NR₅—; R₅ represents a hydrogen atom, a substituted or unsubstituted alkyl group, or a substituted or unsubstituted aryl group; R₄ represents a substituted or unsubstituted alkyl group, a substituted or unsubstituted aryl group, or a substituted or unsubstituted heteroaryl group; a represents an integer of 0 to 3; and plural Z's may be the same as or different from each other).

[2] The organic non-linear optical material according to [1],
in which A₁ represents a phenylene group, a naphthylene group, a divalent thiophene ring (thienylene group), a divalent pyrrole ring, or a divalent furan ring.

[3] The organic non-linear optical material according to [1] or [2],
in which Formula (I) is represented by the following Formula (III):

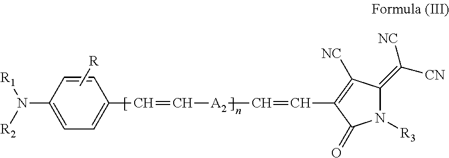

(wherein R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₂ represents an aromatic group; n represents an integer of 0 to 2; plural A₂'s may be the same as or different from each other; R has 3 to 30 carbon atoms and is represented by the Formula (II); and R may be singular or plural, and plural R's may be the same as or different from each other).

[4] The organic non-linear optical material according to any one of [1] to [3],
in which in Formula (I) or (III), A₂ represents a substituted or unsubstituted phenylene group, a substituted or unsubstituted thienylene group, a substituted or unsubstituted divalent pyrrole ring, or a substituted or unsubstituted divalent thiazole ring.

[5] The organic non-linear optical material according to any one of [1] to [3],
in which in Formula (I) or (III), R has 3 to 30 carbon atoms and represents a substituted or unsubstituted alkyl group, a substituted or unsubstituted alkoxy group, a substituted or unsubstituted aryloxy group, a substituted or unsubstituted alkylthio group, a substituted or unsubstituted arylthio group, or a substituted or unsubstituted acylamino group.

[6] An optical element including the organic non-linear optical material according to any one of [1] to [5].

[7] An optical modulator including the organic non-linear optical material according to any one of [1] to [5].

[8] A compound represented by the following Formula (I):

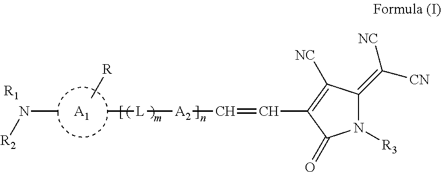

(wherein R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₁ and A₂ each independently represents an aromatic group; L represents —CR₆═CR₇—, —C≡C—, —N═CR₈—, or —CR₉═N— (R₆, R₇, R₈, and R₉ each independently represents a hydrogen atom, a substituted or unsubstituted alkyl group, or a substituted or unsubstituted aryl group); m represents an integer of 0 or 1; n represents an integer of 0 to 2; plural L's, A₂'s and m's may be the same as or different from each other; R has 3 to 30 carbon atoms and is represented by the following Formula (II); and R may be singular or plural, and plural R's may be the same as or different from each other):

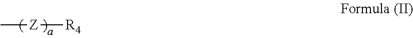

(wherein Z represents —O—, —S—, —CO—, —SO—, —SO₂—, or —NR₅—; R₅ represents a hydrogen atom, a substituted or unsubstituted alkyl group, or a substituted or unsubstituted aryl group; R₄ represents a substituted or unsubstituted alkyl group, a substituted or unsubstituted aryl group, or a substituted or unsubstituted heteroaryl group; a represents an integer of 0 to 3; and plural Z's may be the same as or different from each other).

The organic non-linear optical material according to the present invention is superior in non-linear optical performance and orientation and contains: an organic compound having superior compatibility with a polymer binder; and a polymer binder. In the organic non-linear optical material according to the present invention, high non-linear optical activity and superior compatibility with a polymer binder having a high glass transition temperature are simultaneously realized. Therefore, the high orientation state of the organic compound having non-linear optical activity can be maintained for a long period of time, and preferable effects such as prevention of bleed-out for a long period of time can be exhibited.

By using the organic non-linear optical material according to the present invention, a non-linear optical element which is superior in various characteristics and stability can be implemented.

### DESCRIPTION OF THE PREFERRED EMBODIMENTS

Hereinafter, the present invention will be described based on a representative embodiment. However, within a range not departing from the scope of the present invention, the present invention is not limited to the embodiment described below.

In this specification, numerical ranges represented by “to” include numerical values before and after “to” as lower limits and upper limits.

An organic non-linear optical material according to the present invention contains a compound represented by the following Formula (I) and a polymer binder,

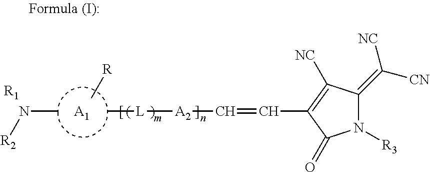

(wherein R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₁ and A₂ each independently represents an aromatic group; L represents —CR₆═CR₇—, —C≡C—, —N═CR₈—, or —CR₉═N— (R₆, R₇, R₈, and R₉ each independently represents a hydrogen atom, a substituted or unsubstituted alkyl group, or a substituted or unsubstituted aryl group); m represents an integer of 0 or 1; n represents an integer of 0 to 2; plural L's, A₂'s and m's may be the same as or different from each other; R has 3 to 30 carbon atoms and is represented by the following Formula (II); and R may be singular or plural, and plural R's may be the same as or different from each other).

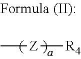

(wherein Z represents —O—, —S—, —CO—, —SO—, —SO₂—, or —NR₅—; R₅ represents a hydrogen atom, a substituted or unsubstituted alkyl group, or a substituted or unsubstituted aryl group; R₄ represents a substituted or unsubstituted alkyl group, a substituted or unsubstituted aryl group, or a substituted or unsubstituted heteroaryl group; a represents an integer of 0 to 3; and plural Z's may be the same as or different from each other).

In the compound represented by Formula (I), R has an effect of inhibiting intermolecular stacking to reduce crystallinity. Therefore, the compound can be stably present in the polymer binder in a state of being dispersed. As a result, an effect of superior compatibility with the polymer binder is exhibited.

In addition, R in the compound represented by Formula (I) prevents the compound from generating an association state accompanying antiparallel orientation. As a result, an effect of increasing the orientation degree of the compound is exhibited. Here, antiparallel orientation refers to an association state between two molecules which is established by Coulomb's force, in which a negative side of a counter molecule is attracted to a positive side of a rod-like molecule having a dipole moment, and a positive side of the counter molecule is attracted to a negative side of the rod-like molecule. As the dipole moment of the compound increases, antiparallel orientation is more likely to occur. When the compound is in an antiparallel orientation state, the dipole moments of the molecules are canceled out. Therefore, the response to electric field poling significantly decreases. In particular, a compound having a substituted amino group as a high electron-donating group and having a tricyanopyrroline skeleton as a high electron-attracting group is likely to cause the above association state.

<Organic Compound having Non-Linear Optical Activity>

The organic non-linear optical material according to the present invention includes: a compound represented by Formula (I) (hereinafter, also referred to as “compound of Formula (I)”) as an organic compound having non-linear optical activity; and a polymer binder. Here, the compound represented by Formula (I) may be dispersed in the polymer binder described below in a microcrystal state or a molecular state or may be chemically linked to a side chain or a main chain of the polymer binder. From the viewpoint of optical quality such as transparency, it is preferable that the compound represented by Formula (I) is dispersed in the polymer binder in a molecular state.

R₁ and R₂ each independently represents a substituted or unsubstituted alkyl group or a substituted or unsubstituted aryl group.

Examples of the alkyl group include a methyl group, an ethyl group, an n-propyl group, an i-propyl group, an n-butyl group, a t-butyl group, an n-hexyl group, a 2-ethylhexyl group, and a t-octyl group. Among these, an ethyl group, an n-propyl group, an n-butyl group, an n-hexyl group, or a 2-ethylhexyl group is preferable; and an ethyl group, an n-butyl group, or an n-hexyl group is more preferable.

Examples of the aryl group include a phenyl group and a naphthyl group. Among these, a phenyl group is preferable.

The alkyl group and the aryl group may further have a substituent. Examples of the substitute include an acyloxy group, an alkoxy group, an aryloxy group, a carbamoyloxy group, an alkylamino group, an anilino group, an acylamino group, a sulfamoyl group, a sulfonyl group, an acyl group, an oxycarbonyl group, a carbamoyl group, a carboxyl group, a hydroxyl group, a silyl group, and a fluorine atom. Among these, an acyloxy group or an alkoxy group is preferable; and an acyloxy group is more preferable. In addition, when the aryl group has a substituent, a ring may be formed, for example, as in carbazole.

Here, the number of carbon atoms in the groups represented by R₁ and R₂ is preferably 2 to 30. When R₁ and R₂ represent a substituted or an unsubstituted alkyl group, the number of carbon atoms is preferably 2 to 20 and more preferably 4 to 20. When R₁ and R₂ represent a substituted or an unsubstituted aryl group, the number of carbon atoms is preferably 6 to 30. When the number of carbon atoms in the groups represented by R₁ and R₂ is 2 or more, the solubility of the compound of Formula (I) in a solvent (solvent used in a coating solution when the organic non-linear optical material is prepared using a wet coating method) increases, and thus uniform coating can be performed. On the other hand, when the number of carbon atoms in the groups represented by R₁ and R₂ is 30 or less, a decrease in the amount of a non-linear optically active component per weight can be suppressed.

R₁ and R₂ each independently represents: preferably, an ethyl group, an n-butyl group, an n-hexyl group, a substituted ethyl group, a substituted butyl group, a substituted hexyl group, or a substituted 2-ethylhexyl group; more preferably, an ethyl group, an n-butyl group, a substituted ethyl group, a substituted butyl group, or a substituted hexyl group; still more preferably, an ethyl group, an n-butyl group, an acyloxy group-substituted ethyl group, an acyloxy group-substituted butyl group, an acyloxy group-substituted hexyl group, an alkoxy group-substituted ethyl group, an alkoxy group-substituted butyl group, or an alkoxy group-substituted hexyl group; and even still more preferably, an ethyl group, an n-butyl group, an acyloxy group-substituted ethyl group, an acyloxy group-substituted butyl group, or an acyloxy group-substituted hexyl group.

In Formula (I), R₃ represents a hydrogen atom, a substituted or unsubstituted alkyl group, a substituted or unsubstituted aryl group, a substituted carbonyl group, or a substituted or unsubstituted sulfonyl group.

From the viewpoints of improving the solubility of the compound and suppressing intermolecular aggregation, R₃ represents: preferably, a hydrogen atom or a substituted or unsubstituted alkyl group having 30 or less carbon atoms; and more preferably, a hydrogen atom or a substituted or unsubstituted alkyl group having 20 or less carbon atoms.

Examples of the alkyl group include the above-described alkyl groups represented by R₁ and R₂. Among these, a methyl group, an ethyl group, an n-propyl group, an i-propyl group, an n-butyl group, a t-butyl group, an n-hexyl group, or a 2-ethylhexyl group, is preferable; an ethyl group, an n-butyl group, or an n-hexyl group is more preferable; and an n-butyl group is still more preferable.

Examples of the aryl group include the above-described aryl groups represented by R₁ and R₂. Among these, a phenyl group is preferable.

When the alkyl group and the aryl group further have a substituent, examples of the substituent include the above-described substituents of the groups represented by R₁ and R₂, and preferable examples thereof are also the same.

When the carbonyl group and the sulfonyl group further have a substituent, examples of the substituent include an alkyl group, an aryl group, an alkoxy group, an aryloxy group, an alkylamino group, and an arylamino group. As the substituent, an alkyl group or an aryl group is preferable, and an alkyl group is more preferable.

In Formula (I), L represents —CR₆═CR₇—, —C≡C—, —N═CR₈—, or —CR₉═N—. R₆, R₇, R₈, and R₉ each independently represents a hydrogen atom, a substituted or unsubstituted alkyl group, or a substituted or unsubstituted aryl group. L represents: preferably, —CR₆═CR₇— or —C≡C—; more preferably, —CR₆═CR₇—; and still more preferably —CH═CH—.

Examples of the alkyl groups represented by R₆, R₇, R₈, and R₉ include the above-described alkyl groups represented by R₁ and R₂. Examples of the aryl groups represented by R₆, R₇, R₈, and R₉ include the above-described aryl groups represented by R₁ and R₂.

When the alkyl groups and the aryl groups represented by R₆, R₇, R₈, and R₉ further have a substituent, examples of the substituent include the above-described substituents of the groups represented by R₁ and R₂, and preferable examples thereof are also the same.

R₆, R₇, R₈, and R₉ each independently represents: preferably, a hydrogen atom, a substituted or unsubstituted alkyl group having 20 or less carbon atoms, or a substituted or unsubstituted aryl group having 6 to 30 carbon atoms; and more preferably, a hydrogen atom.

From the viewpoints of contributing to the π conjugated system extension of the compound and improving non-linear optical characteristics, it is preferable that —CH═CH— is introduced into a linking portion between an oxopyrroline ring and A₂. However, even when plural —CH═CH—'s are linked and introduced, the above-described effect is not enhanced. Therefore, it is preferable that —CH═CH— is singular. When —CH═CH— is introduced, trans and cis isomers are present. However, it is preferable that only trans isomers are present from the viewpoint of effectively extending the π conjugated system. When —CH═CH— is singular, substantially only trans isomers are stably present three-dimensionally. When plural —CH═CH—'s are linked and introduced, the proportion of cis isomers increases.

In Formula (I), R represents a substituent with which A₁ is substituted. R has 3 to 30 carbon atoms and is represented by the following Formula (II). R may be singular or plural, and plural R's may be the same as or different from each other. It is preferable that the number of R's with which A₁ is substituted is one or two.

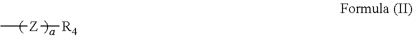

(wherein Z represents —O—, —S—, —CO—, —SO—, —SO₂—, or —NR₅—; R₅ represents a hydrogen atom, a substituted or unsubstituted alkyl group, or a substituted or unsubstituted aryl group; R₄ represents a substituted or unsubstituted alkyl group, a substituted or unsubstituted aryl group, or a substituted or unsubstituted heteroaryl group; a represents an integer of 0 to 3; and plural Z's may be the same as or different from each other).

Z represents —O—, —S—, —CO—, —SO—, —SO₂—, or —NR₅—; preferably —O—, —O—, —CO—, or —NR₅—; and more preferably —O—. From the viewpoint of improving the non-linear optical activity of the compound, it is preferable that R represents an electron-donating group and that Z represents a substituent such that R represents an electron-donating group.

R₅ represents a hydrogen atom, a substituted or unsubstituted alkyl group, or a substituted or unsubstituted aryl group; and preferably, a hydrogen atom or a substituted or unsubstituted alkyl group.

Examples of the alkyl group and the aryl group represented by R₅ include the above-described alkyl groups and aryl groups represented by R₁ and R₂.

R₄ represents: a substituted or unsubstituted alkyl group, a substituted or unsubstituted aryl group, or a substituted or unsubstituted heteroaryl group; preferably, a substituted or unsubstituted alkyl group or a substituted or unsubstituted aryl group; and more preferably a substituted or unsubstituted alkyl group.

Examples of the alkyl group represented by R₄ include linear or branched alkyl groups having 1 to 30 carbon atoms. Among these, an alkyl group having 3 to 30 carbon atoms is preferable, and an alkyl group having 3 to 15 carbon atoms is more preferable. In order to reduce the crystallinity of the compound, as the substituent represented by R₄ becomes more bulky, the effect thereof increases. However, when the substituent is extremely bulky, the non-linear optical characteristics of the compound per mass deteriorate, and it is preferable that the substituent is an alkyl group having 30 or less carbon atoms.

It is preferable that R has 3 to 30 carbon atoms and represents a substituted or unsubstituted alkyl group, a substituted or unsubstituted alkoxy group, a substituted or unsubstituted aryloxy group, a substituted or unsubstituted alkylamino group, a substituted or unsubstituted acylamino group, a substituted or unsubstituted alkylthio group, or a substituted or unsubstituted arylthio group. It is more preferable that R has 3 to 30 carbon atoms and represents a substituted or unsubstituted alkyl group, a substituted or unsubstituted alkoxy group, a substituted or unsubstituted aryloxy group, a substituted or unsubstituted alkylthio group, a substituted or unsubstituted arylthio group, or a substituted or unsubstituted acylamino group. It is still more preferable that R has 3 to 15 carbon atoms and represents a substituted or unsubstituted alkyl group, a substituted or unsubstituted alkoxy group, or a substituted or unsubstituted acylamino group.

In Formula (II), a represents an integer of 0 to 3. It is preferable that a represents an integer of 0 to 2.

In Formula (I), A₁ and A₂ each independently represents an aromatic group. Examples of the aromatic group include a phenylene group and a naphthylene group. In addition, A₁ and A₂ may each independently represent a heterocyclic aromatic group.

It is preferable that the heterocyclic aromatic group is a 5-membered or 6-membered heterocyclic aromatic group and that a heteroatom of the ring configuration is an oxygen atom, a sulfur atom, or a nitrogen atom. It is more preferable that the heterocyclic aromatic group is a 5-membered or 6-membered heterocyclic aromatic group having 3 to 30 carbon atoms and that a heteroatom of the ring configuration is a sulfur atom or a nitrogen atom.

Examples of the heterocyclic aromatic group include divalent rings including a pyridine ring, a pyrazine ring, a pyridazine ring, a pyrimidine ring, a triazine ring, a quinoline ring, an isoquinoline ring, a quinazoline ring, a cinnoline ring, a phthalazine ring, a quinoxaline ring, a pyrrole ring, an indole ring, a furan ring, a benzofuran ring, a thiophene ring, a benzothiophene ring, a pyrazole ring, an imidazole ring, a benzimidazole ring, a triazole ring, an oxazole ring, a benzoxazole ring, a thiazole ring, a benzothiazole ring, an isothiazole ring, a benzisothiazole ring, a thiadiazole ring, an isoxazole ring, and a benzisoxazole ring.

The aromatic group represented by A₂ may further have a substituent, and examples of the substituent include an alkyl group, an aryl group, a heteroaryl group, an alkoxy group, an acyloxy group, a carbamoyloxy group, an alkylamino group, an alkylthio group, an anilino group, an acylamino group, a sulfamoyl group, a sulfonyl group, an acyl group, an oxycarbonyl group, a carbonyl group, a carbamoyl group, a carboxyl group, a cyano group, a nitro group, a sulfo group, and a halogen atom.

A₁ represents: preferably, a phenylene group, a naphthylene group, a divalent thiophene ring (thienylene group), a divalent pyrrole ring, or a divalent furan ring; and more preferably, a phenylene group, a divalent thiophene ring (thienylene group), a divalent pyrrole ring, or a divalent furan ring.

A₂ represents: preferably, a substituted or unsubstituted phenylene group, a substituted or unsubstituted thiophene ring (thienylene group), a substituted or unsubstituted divalent pyrrole ring, or a substituted or unsubstituted divalent thiazole ring; more preferably, a substituted or unsubstituted thienylene group, a substituted or unsubstituted divalent thiazole ring, or a substituted or unsubstituted phenylene group; and still more preferably a substituted or unsubstituted phenylene group or a substituted or unsubstituted thienylene group.

In addition, when A₂ has a substituent, the substituent is: preferably a halogen atom, a cyano group, a substituted or unsubstituted alkyl group, a substituted or unsubstituted aryl group, a substituted or unsubstituted heteroaryl group, a substituted or unsubstituted alkoxy group, a substituted carbonyl group, or a substituted or unsubstituted carbamoyl group; more preferably, a cyano group, a substituted or unsubstituted alkyl group, a substituted or unsubstituted aryl group, a substituted or unsubstituted alkoxy group, or a substituted carbonyl group; and still more preferably, a cyano group, a substituted or unsubstituted alkyl group, or a substituted or unsubstituted aryl group.

In Formula (I), m represents an integer of 0 or 1. m is preferably 1 from the viewpoint of non-linear optical activity, and m is preferably 0 from the viewpoint of solubility because crystallinity is reduced.

In Formula (I), n represents an integer of 0 to 2. It is preferable that n represents 0 or 1.

The present invention relates to the compound represented by Formula (I).

The compound represented by Formula (I) has non-linear optical activity and thus is useful as a non-linear optical material.

It is more preferable that the compound represented by Formula (I) is a compound represented by the following Formula (Ia).

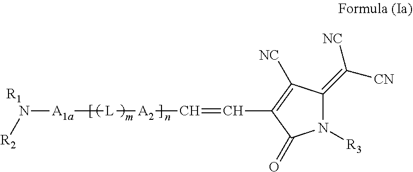

(in Formula (Ia), R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₁ₐ represents any one of the following linking groups (a1) to (a4); A₂ represents an aromatic group; L represents —CR₆═CR₇—, —C≡C—, —N═CR₈—, or —CR₉═N— (R₆, R₇, R₈, and R₉ each independently represents a hydrogen atom, a substituted or unsubstituted alkyl group, or a substituted or unsubstituted aryl group); m represents an integer of 0 or 1; n represents an integer of 0 to 2; and plural L's, A₂'s and m's may be the same as or different from each other).

In Formula (Ia), R₁, R₂, R₃, A₂, m, and n have the same definitions as R₁, R₂, R₃, A₂, m, and n in Formula (I), and preferable ranges thereof are also the same.

A₁ₐ represents any one of the following linking groups (a1) to (a4).

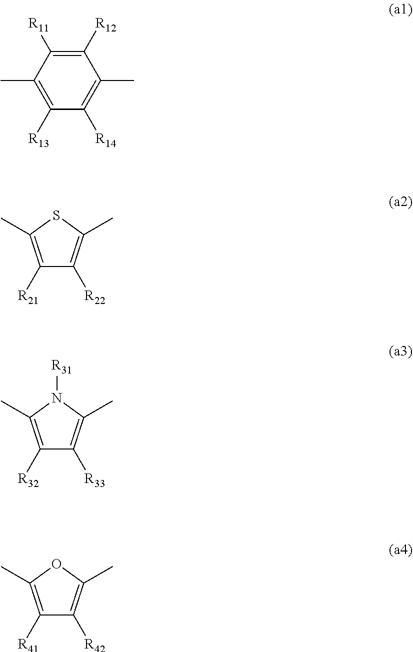

(R₁₁ to R₁₄, R₂₁, R₂₂, R₃₁ to R₃₃, R₄₁, and R₄₂ each independently represents a hydrogen atom or a substituent having 3 to 30 carbon atoms and represented by Formula (II), in which all of R₁₁, R₁₂, R₁₃, and R₁₄ do not represent a hydrogen atom at the same time, and both of R₂₁ and R₂₂, all of R₃₁, R₃₂, and R₃₃, or both of R₄₁ and R₄₂ do not represent a hydrogen atom at the same time).

It is more preferable that the compound represented by Formula (I) is a compound represented by the following Formula (III).

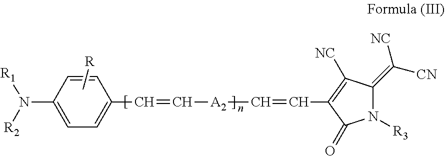

(wherein R₁ and R₂ each independently represents a substituted or unsubstituted alkyl group or a substituted or unsubstituted aryl group; R₃ represents a hydrogen atom, a substituted or unsubstituted alkyl group, a substituted or unsubstituted aryl group, a substituted carbonyl group, or a substituted or unsubstituted sulfonyl group; A₂ represents an aromatic group; n represents an integer of 0 to 2; plural A₂'s may be the same as or different from each other; R has 3 to 30 carbon atoms and is represented by Formula (II); and R may be singular or plural, and plural R's may be the same as or different from each other).

In Formula (III), R₁, R₂, R₃, R, A₂, and n have the same definitions as R₁, R₂, R₃, R, A₂, and n in Formula (I), and preferable ranges thereof are also the same.

Hereinafter, specific examples of the organic compounds having non-linear optical activity represented by Formulae (I) to (III) which are preferably used in the present invention will be shown. However, the scope of the present invention is not limited to these examples.

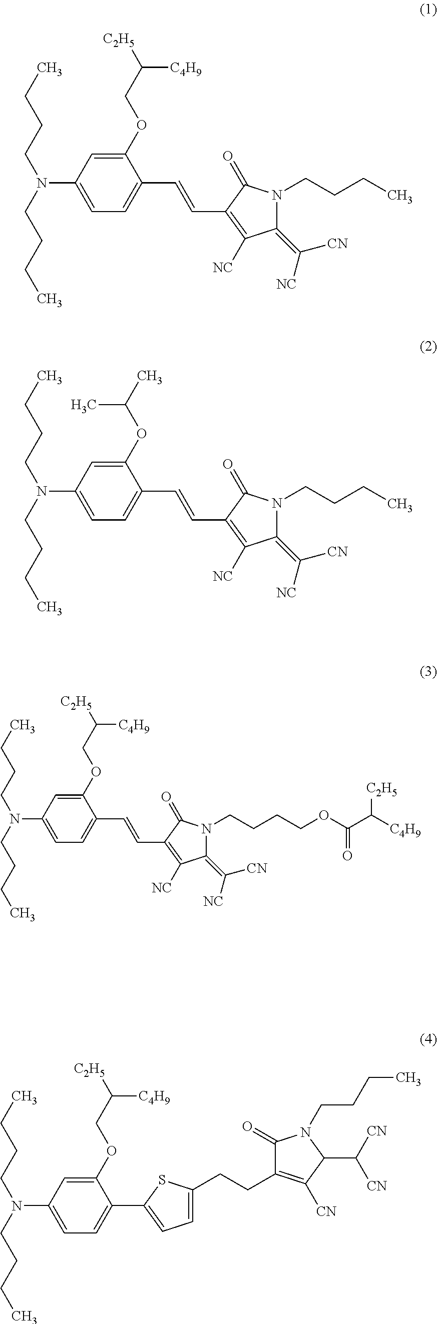

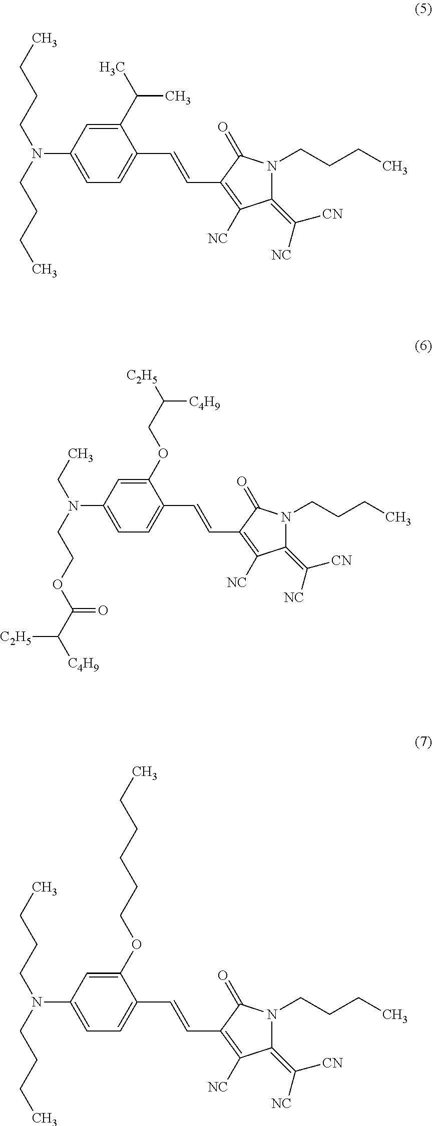

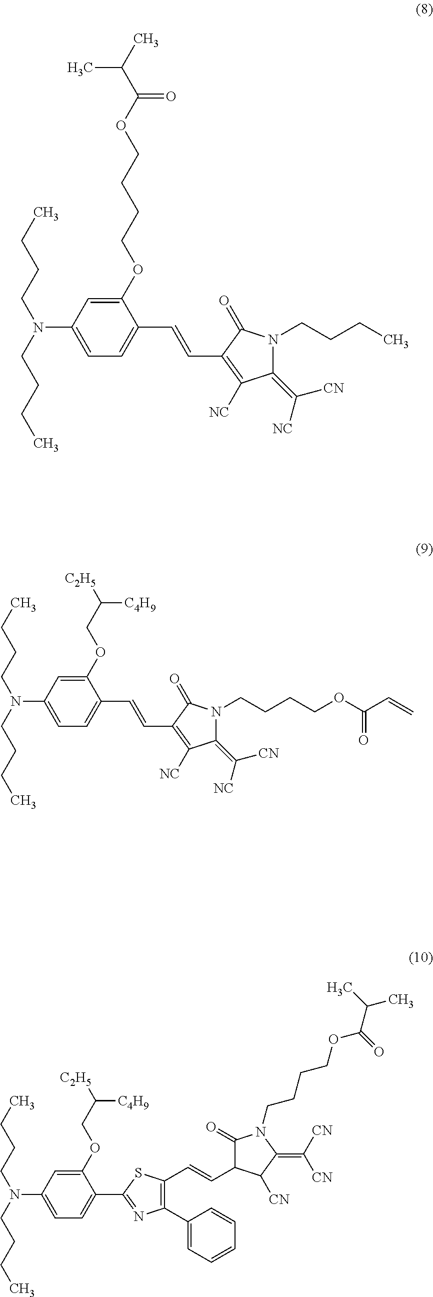

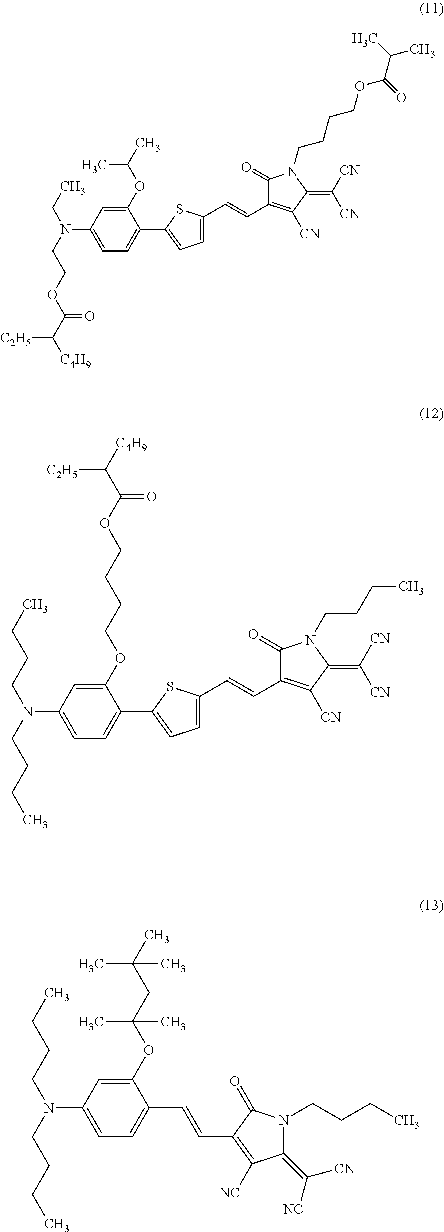

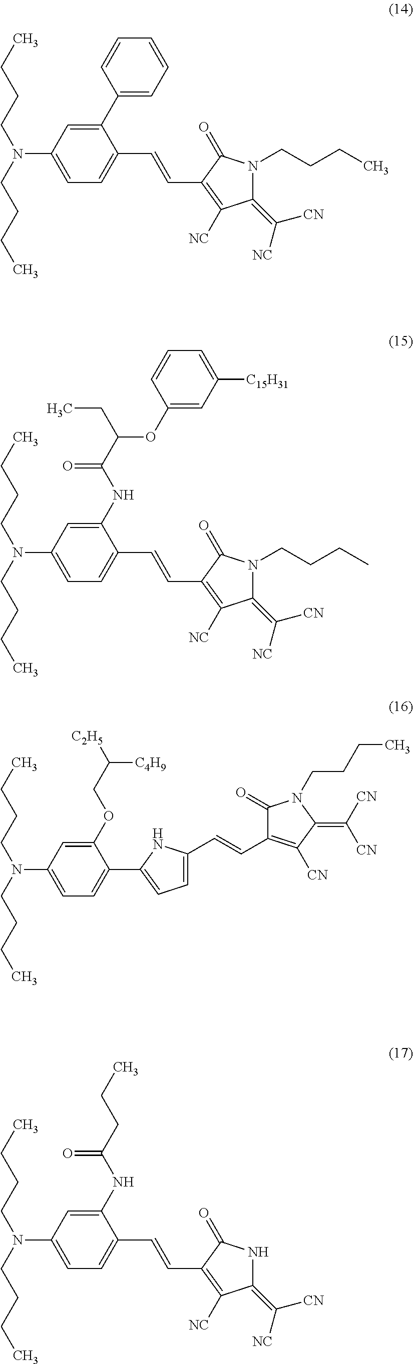

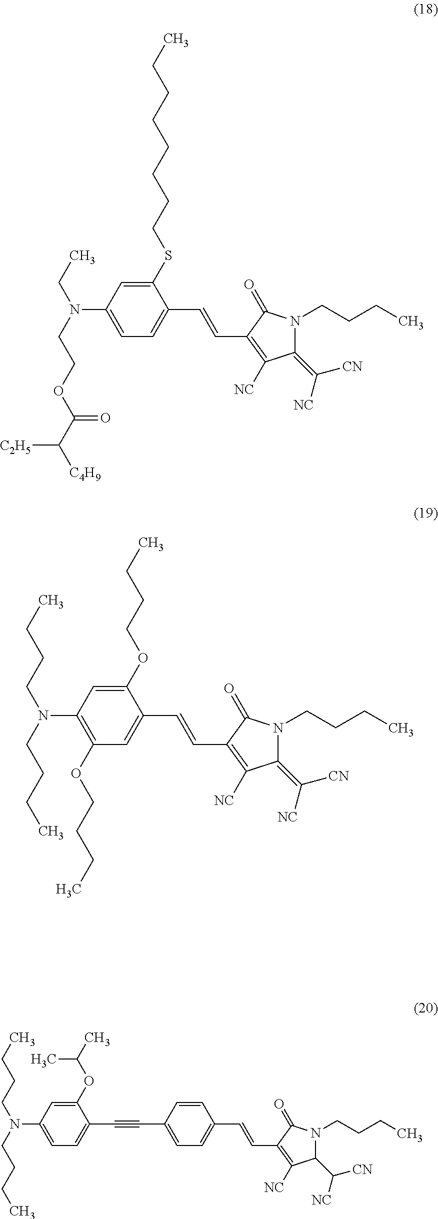

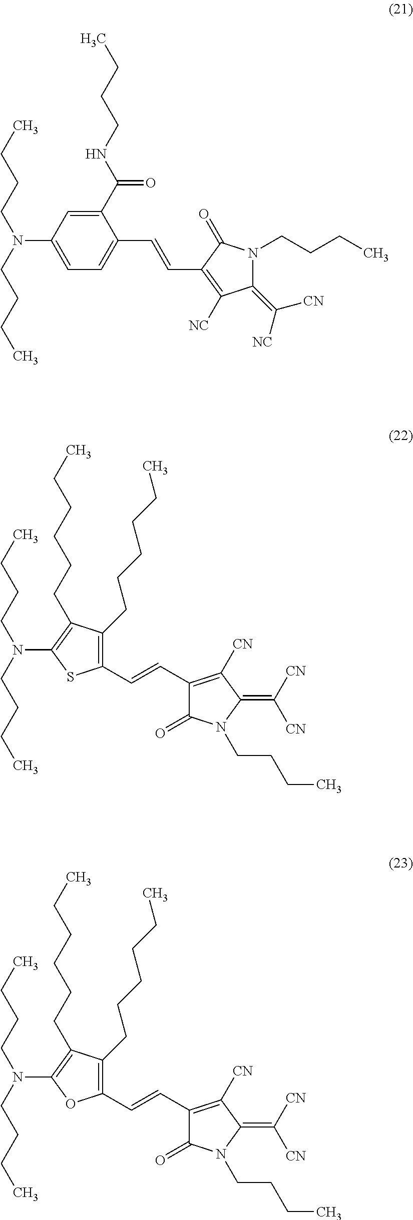 [SMILES: CCCCNC(=O)c1cc(N(CCCC)CCCC)ccc1/C=C/C1=C(C#N)C(=C(C#N)C#N)N(CCCC)C1=O ; CCCCCCc1c(/C=C/C2=C(C#N)C(=C(C#N)C#N)N(CCCC)C2=O)sc(N(CCCC)CCCC)c1CCCCCC ; CCCCCCc1c(/C=C/C2=C(C#N)C(=C(C#N)C#N)N(CCCC)C2=O)oc(N(CCCC)CCCC)c1CCCCCC]

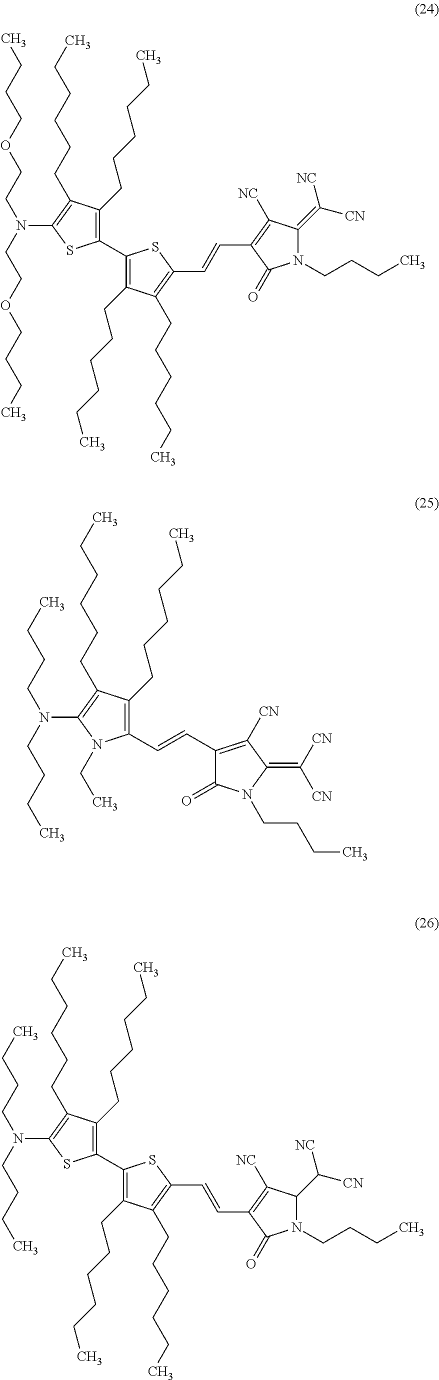 [SMILES: CCCCCCc1c(/C=C/C2=C(C#N)C(=C(C#N)C#N)N(CCCC)C2=O)sc(-c2sc(N(CCOCCCC)CCOCCCC)c(CCCCCC)c2CCCCCC)c1CCCCCC ; CCCCCCc1c(CCCCCC)c(N(CCCC)CCCC)n(CC)c1/C=C/C1=C(C#N)C(=C(C#N)C#N)N(CCCC)C1=O ; CCCCCCc1c(/C=C/C2=C(C#N)C(C(C#N)C#N)N(CCCC)C2=O)sc(-c2sc(N(CCCC)CCCC)c(CCCCCC)c2CCCCCC)c1CCCCCC]

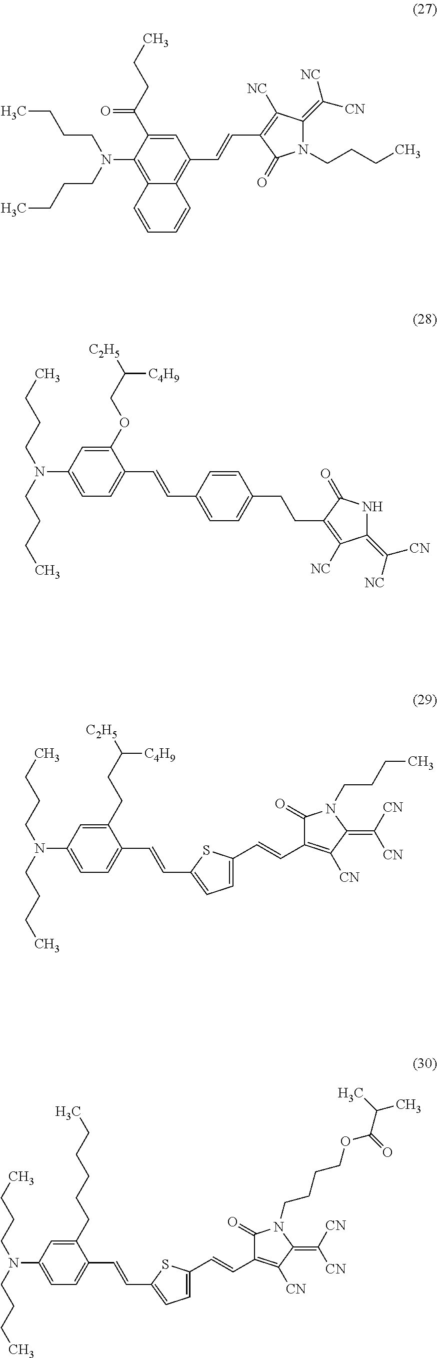

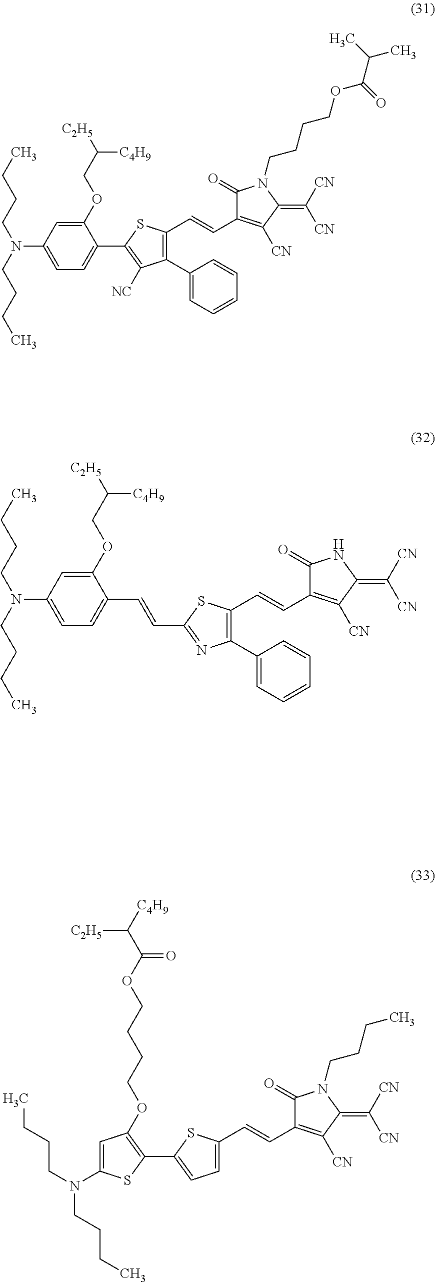

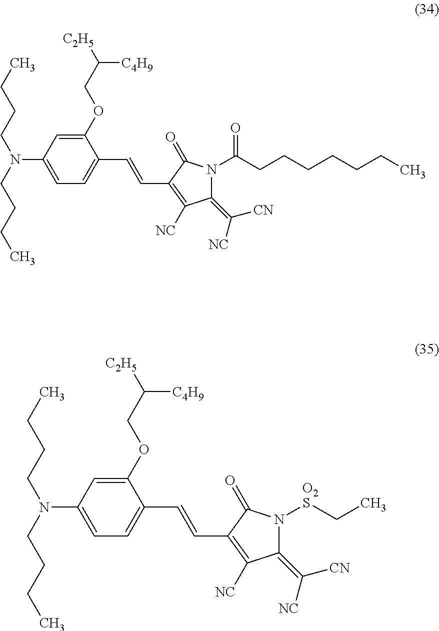

Hereinafter, a synthesis method of the organic compound having non-linear optical activity used in the present invention will be described. The organic compound having non-linear optical activity used in the present invention is synthesized through a condensation reaction between a TCP acceptor and aldehyde corresponding thereto, for example, as in a method described in U.S. Pat. No. 7,307,173B. The corresponding aldehyde can be synthesized, for example, using the Vilsmeier reaction described on page 668 of “New Experiment Chemistry Course”.

The details of specific examples of the reaction will be described below in Examples.

The sublimation temperature of the above-described organic compound having non-linear optical activity used in the present invention is preferably 130° C. or higher and more preferably 170° C. or higher.

In addition, as described above, it is necessary that the organic compound having non-linear optical activity used in the present invention has superior solubility in a solvent of a coating solution which is used for preparing the organic non-linear optical material. Regarding the solubility, for example, preferably 1 mass % or higher and more preferably 5 mass % or higher of the organic compound is dissolved in a solvent such as tetrahydrofuran, cyclopentanone, chloroform, or N,N-dimethylacetamide at room temperature.

Further, the electro-optic constant of the organic compound having non-linear optical activity used in the present invention is mainly in proportion to a hyperpolarizability β₀ of the organic compound having non-linear optical activity in an electrostatic field. Therefore, β₀ is preferably 150×10⁻³⁰ D·esu or higher and more preferably 200×10⁻³⁰ D·esu or higher. β₀ can be estimated using commercially available molecular orbital calculation simulation software.

In an optical modulator according to the present invention, the organic non-linear optical material according to the present invention can be used. As the electro-optic constant of the non-linear optical material constituting the optical modulator increases, the size and driving voltage of the modulator can be reduced. In a use wavelength of the modulator, the electro-optic constant is preferably 5 pm/V or higher and more preferably 7 pm/V or higher. The electro-optic constant can be measured using a typical measurement method such as an ATR method, ellipsometry, or a prism coupler method.

In the organic non-linear optical material according to the present invention, although the content of the organic compound having non-linear optical activity varies depending on the required non-linear optical performance and mechanical strength, the kind of the organic compound having non-linear optical activity to be used, and the like, in general, a ratio of the mass of the organic compound to the total mass of the organic non-linear optical material is preferably within a range of 1 mass % to 90 mass %. The reason for this is as follows. When the ratio is 1 mass % or higher, non-linear optical performance can be obtained. In addition when the ratio is 90 mass % or less, a problem such as insufficient mechanical strength can be prevented. The content of the organic compound having non-linear optical activity is more preferably within a range of 5 mass % to 75 mass % and still more preferably within a range of 10 mass % to 60 mass %.

The preferable content of the organic compound having non-linear optical activity is within the same range irrespective of whether the organic compound having non-linear optical activity is dispersed in or bonded to the polymer binder.

<Polymer Binder>

The polymer binder used in the present invention is not particularly limited as long as it is superior in optical quality and film formability. From the viewpoint of suppressing the orientation relaxation of the organic compound having non-linear optical activity, it is preferable that the polymer binder has a glass transition temperature of 130° C. or higher. It is more preferable that the polymer binder has a glass transition temperature of 140° C. or higher and high mechanical strength. Specific examples of the polymer binder include polycarbonate, polyimide, polyarylate, polycyclic olefin, polycyanurate, polyester, acrylic polymer, and epoxy polymer. In addition, a mixture or a copolymer including two or more polymers among the above plural polymers may be used.

In the present invention, the glass transition temperatures of the polymer binder and the organic non-linear optical material described below are measured using a differential scanning calorimeter (DSC), and when the temperature is increased by 10° C. per minute from room temperature, a temperature corresponding to an intersection between a baseline and a slope of a rising portion in an endothermic process accompanied by glass transition is set as a glass transition temperature.

In the organic non-linear optical material according to the present invention, a ratio of the content of the organic compound having non-linear optical activity to the content of the polymer binder is preferably 1/99 to 90/10 and more preferably 5/95 to 60/40.

<Other Components>

In addition to the organic compound having non-linear optical activity and the polymer binder, optionally, other additives can be added to the organic non-linear optical material according to the present invention. For example, in order to suppress the oxidation of the organic compound having non-linear optical activity and/or the polymer binder, a well-known antioxidant such as 2,6-di-t-butyl-4-methylphenol or hydroquinone may be used. In addition in order to suppress the deterioration of the organic compound having non-linear optical activity and/or the polymer binder caused by ultraviolet rays, a well-known ultraviolet absorber such as 2,4-dihydroxybenzophenone or 2-hydroxy-4-methoxybenzophenone may be used. In addition, as a refractive index regulator for improving performance as an optical element, inorganic particles (for example, zirconium oxide, titanium oxide, or zinc sulfide) or a high-refractive-index organic compound (for example, diphenyl sulfide, diphenyl, or diphenyl sulfoxide) can be used.

When the above-described additives are added, it is preferable that the content of the polymer binder including the organic compound having non-linear optical activity, which is configured to have the above-described preferable content ratio, is 1 part by mass to 99 parts by mass and that the content of the additives is 1 part by mass to 99 parts by mass, and it is more preferable that the content of the polymer binder including the organic compound having non-linear optical activity is 5 parts by mass to 90 parts by mass and that the content of the additives is 10 parts by mass to 95 parts by mass.

In addition, when the organic non-linear optical material is prepared using a wet coating method, a well-known leveling agent such as silicone oil may be added to a coating solution in order to improve the surface smoothness of a coating film. Alternatively, when an organic compound having non-linear optical activity and/or a polymer binder which has a crosslinking curable functional group is used, a well-known curing catalyst or auxiliary curing agent may be added to promote crosslinking curing.

<Organic Non-Linear Optical Material>

The form of the organic non-linear optical material is not particularly limited but is generally in the form of a thin film when applied to a non-linear optical element. As a method of forming a thin film containing the organic non-linear optical material according to the present invention, a well-known method such as an injection molding method, a press molding method, a soft lithography method, or a wet coating method can be used. However, from the viewpoints of the simplicity, mass productivity, film quality (for example, uniformity in film thickness or reduction in defects such as bubbles), and the like of the manufacturing device, a wet coating method is preferable in which a solution obtained by dissolving at least the organic compound having non-linear optical activity and the polymer binder in an organic solvent is applied to an appropriate substrate using a method such as a spin coating method, a blade coating method, a dip coating method, an ink jet method, or a spray coating method.

The organic solvent used in the wet coating method is not particularly limited as long as the organic compound having non-linear optical activity and the polymer binder to be used can be dissolved therein, and it is preferable that the organic solvent has a melting point of 80° C. to 200° C. When an organic solvent having a melting point of lower than 80° C. is used, there are problems in that, for example, the viscosity of the coating solution may be changed (increased) due to the volatilization of the solvent when the coating solution is stored, or condensation may occur due to an extremely high volatilization speed of the solvent when the coating solution is applied. On the other hand, when an organic solvent having a melting point of higher than 200° C. is used, it is difficult to remove the solvent after the application, and thus there are problems in that, for example, the remaining organic solvent functions as a plasticizer of the polymer binder so as to cause a decrease in the glass transition temperature.

Preferable examples of the organic solvent include diethylene glycol dimethyl ether, cyclopentanone, cyclohexanone, cyclohexanol, toluene, chlorobenzene, xylene, N,N-dimethylformamide, N,N-dimethylacetamide, dimethyl sulfoxide, 2,2,3,3-tetrafluoro-1-propanol, 1,2-dichloroethane, 1,2-dichloropropane, 1,3-dichloropropane, and 1,2,3-trichloropropane. Among these organic solvents, one kind may be used alone, or a mixture of plural kinds may be used. In addition, a mixed solvent obtained by adding an organic solvent having a melting point of lower than 80° C. such as tetrahydrofuran, methyl ethyl ketone, isopropanol, or chloroform to the above-described preferable organic solvents can also be used.

The organic non-linear optical material according to the present invention can be prepared by forming a thin film, for example, with the above-described spin coating method using the above-described coating solution. As described above, a polymer binder having a relatively high glass transition temperature is used as the polymer binder according to the present invention. In addition, from the viewpoints of heat resistance and the like, it is also preferable that the organic non-linear optical material including the prepared organic compound having non-linear optical activity has a high glass transition temperature.

Accordingly, the glass transition temperature of the organic non-linear optical material is preferably 130° C. or higher and more preferably 140° C. or higher.

In order to develop secondary non-linear optical activity in the polymer-based non-linear optical material, as described above, it is necessary that the organic compound having non-linear optical activity is oriented. For example, an orientation method for this includes: applying the polymer-based non-linear optical material to a substrate on which an alignment film is formed; and inducing the orientation of the organic compound having non-linear optical activity in the polymer-based non-linear optical material due to the orientation of the alignment film. In addition, a well-known poling method such as an optical poling method, a light-assisted electric field poling method, or an electric field poling method can be efficiently used. Among these, an electric field poling method is particularly preferable from the viewpoints of the simplicity of the device, the height of the obtained orientation degree, and the like.

The electric field poling method is roughly classified into: a contact poling method of interposing the non-linear optical material between a pair of electrodes to apply an electric field; and a corona poling method of performing corona discharge on a surface of the non-linear optical material on a substrate electrode to apply a charge electric field. The electric field poling method is an orientation method of orienting (poling) the non-linear optically active compound in an applied electric field direction due to Coulomb's force between the dipole moment of the non-linear optically active compound and the applied electric field.

In the electric field poling method, generally, the non-linear optical material is heated to a temperature near the glass transition temperature thereof in a state where an electric field is applied thereto. As a result, the transfer of the orientation of the non-linear optically active compound in the electric field direction is promoted to sufficiently induce orientation, the non-linear optically active compound is cooled to room temperature in a state where an electric field is applied to freeze the orientation state, and then the applied electric field is removed. However, this orientation state is basically a thermodynamic non-equilibrium state and thus becomes randomized gradually over time even at a glass transition temperature or lower. Therefore, there is a fundamental problem in that non-linear optical activity deteriorates.

The larger the difference between the temperature of an environment where the non-linear optical material is disposed and the glass transition temperature, the more gradually the randomization of the orientation state over time progresses. Therefore, by designing the glass transition temperature of the non-linear optical material to be high using a binder resin having a high glass transition temperature, this problem can be solved in practice during actual use. In the present invention, a polymer binder having a glass transition temperature of 150° C. or higher is preferably used. In this case, since the sublimation temperature of the organic compound having non-linear optical activity used in the present invention is high as described above, a non-linear optical material having superior non-linear optical performance and stability can be prepared without being sublimated or deteriorating during heating.

As an index for determining whether or not poling is performed, a numerical value (order parameter: φ) indicating the degree to which non-linear optical molecules (in general, dichroic molecules) are oriented in the electric field direction is used. Specifically, φ can be calculated from “1−(Aₜ/A₀)” in which A₀ represents absorbance when the orientation of the molecules is randomized, and Aₜ represents absorbance when the molecules are oriented in an electric field direction (film thickness direction).

The order parameter is a numerical value of 1 in a theoretical state where all the molecules are completely oriented and is a numerical value of 0 in a state where all the molecules are completely randomized. A high value of the order parameter represents that the overall orientation degree of the molecules is high. By measuring this value, the efficiency of poling can be determined, and stability and the like can be evaluated.

<Optical Element>

An optical element according to the present invention is characterized in that the organic non-linear optical material according to the present invention is used. The optical element is not particularly limited as long as it operates based on a non-linear optical effect, and specific examples thereof include a wavelength conversion element, a photorefractive element, and an electro-optic element. In particular, an electro-optic element such as an optical switch, an optical modulator, or a phase shifter which operates based on an electro-optic effect is preferable.

As the electro-optic element, an element having a structure in which the non-linear optical material is formed on a substrate and is interposed between a pair of electrodes for an electric input signal is preferably used.

As a material constituting the substrate, metal such as aluminum, gold, iron, nickel, chromium, or titanium; a semiconductor such as silicon, titanium oxide, zinc oxide, or gallium arsenide; glass; or a plastic such as polyethylene terephthalate, polyethylene naphthalate, polycarbonate, polysulfone, polyether ketone, or polyimide can be used.

A conductive film may be formed on a surface of the substrate material. As a material of the conductive film, metal such as aluminum, gold, nickel, chromium, or titanium; a conductive oxide such as tin oxide, indium oxide, a composite oxide of tin oxide and indium oxide (ITO), or a composite oxide of indium oxide and zinc oxide (IZO); or a conductive polymer such as polythiophene, polyaniline, polyparaphenylene vinylene, or polyacetylene is used. This conductive film is formed using a well-known dry film formation method such as vapor deposition or sputtering or using a well-known wet film formation method such as dip coating or electrolytic deposition. Optionally, a pattern is formed on the conductive film. A conductive substrate or the above-described conductive film formed on the substrate is used as an electrode (hereinafter, abbreviated as “lower electrode”) during poling or during operation of an element.

On the surface of the substrate, optionally, an adhesion layer for improving adhesion between the substrate and a film formed on the substrate, a leveling layer for smoothing the roughness of the substrate surface, or an interlayer for collectively providing the above functions may be further formed. A material for forming the film is not particularly limited, and examples thereof include well-known materials including: acrylic resins, methacrylic resins, amide resins, vinyl chloride resins, vinyl acetate resins, phenol resins, urethane resins, vinyl alcohol resins, acetal resins, and copolymers thereof; and crosslinked products such as a zirconium chelate compound, a titanium chelate compound, or a silane coupling agent and co-crosslinked products thereof.

It is preferable that the electro-optic element which is the non-linear optical element according to the present invention has a waveguide structure, and it is more preferable that a core layer of the waveguide contains the non-linear optical material according to the present invention.

A cladding layer (hereinafter, abbreviated as “lower cladding layer”) may be formed between the core layer, which contains the non-linear optical material according to the present invention, and the substrate. The lower cladding layer is not particularly limited as long as it has a lower refractive index than the core layer and is not impregnated with the core layer during the formation of the core layer. As the lower cladding layer, for example, an UV-curable or thermosetting resin such as an acrylic resin, an epoxy resin, an oxetane resin, a thiirane resin, or a silicone resin; polyimide; or glass is preferably used.

After forming the core layer using the non-linear optical material according to the present invention, a cladding layer (hereinafter, abbreviated as “upper cladding layer”) may be further formed on the core layer using the same method as in the lower cladding layer. As a result, a slab waveguide having a configuration of substrate/lower cladding layer/core layer/upper cladding layer is formed.

After forming the core layer, the core layer can also be patterned with a well-known method using a semiconductor process technique such as reactive ion etching (RIE), photolithography, or electron beam lithography to form a channel waveguide or ridge waveguide. Alternatively, a portion of the core layer may be patterned by irradiation, for example, UV rays or electron beams to form a channel waveguide in which the refractive index of the irradiated portion is changed.

An electrode (for example, abbreviated as “upper electrode”) for applying an electric input signal to the surface of the upper cladding layer can be formed on a desired region of the upper cladding layer, thereby forming a fundamental electro-optic element.

When the channel waveguide or the ridge waveguide is formed as described above, a well-known device structure such as a linear type, a Y-branched type, a directional coupler type, or a Mach-Zehnder type can be adopted as the pattern of the core layer, and can be applied to a well-known optical information communication device such as an optical switch, an optical modulator, or a phase shifter.

### EXAMPLES

Hereinafter, examples of the present invention will be described, but the present invention is not limited to these examples. “%” and “part(s)” representing content ratios in Examples represent “% by mass” and “part(s) by mass”.

### Synthesis Example 1

### Synthesis of Exemplary Compound (1)

Exemplary Compound (1) was synthesized according to the following scheme.

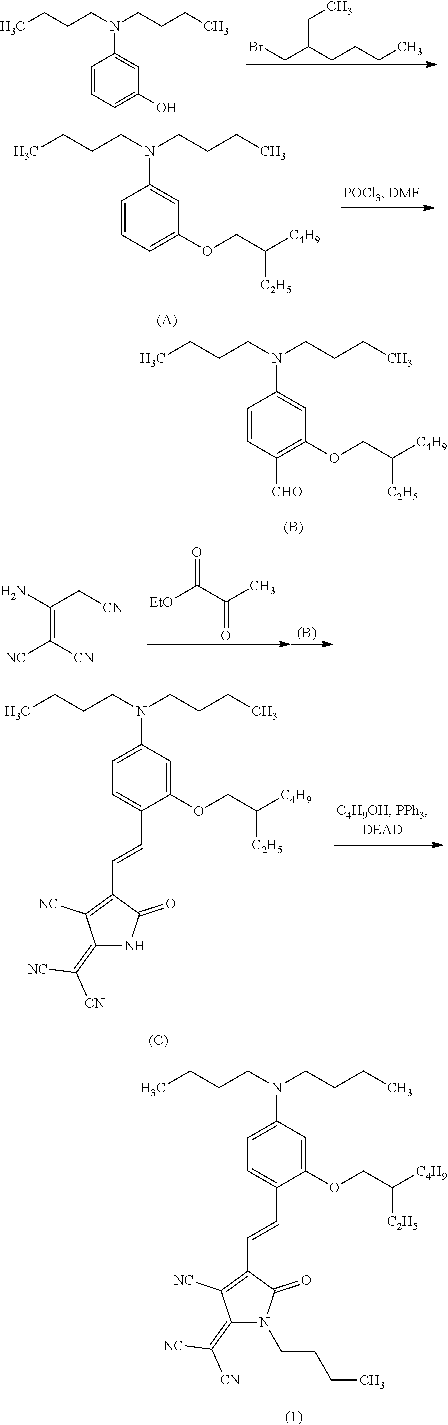

—Synthesis of Intermediate Product (A)—

80 ml of N,N-dimethylacetamide was added to 5 g (0.023 mol) of N,N-dibutyl-3-aminophenol, 5.2 ml (0.030 mol) of 2-ethylhexyl bromide, and 6.1 g (0.044 mol) of potassium carbonate. The obtained solution was heated and stirred at 120° C. for 10 hours under nitrogen flowing conditions. After allowing the solution to cool to room temperature, the solid content was separated by filtration, and 400 ml of ethyl acetate and 400 ml of water were added, followed by liquid separation. After being dehydrated with magnesium sulfate, the solution was filtered, and the solvent of the organic layer was removed by distillation under reduced pressure. The residue was purified using silica gel column chromatography (ethyl acetate/n-hexane=1/9). As a result, 4.8 g of Intermediate Product (A) was obtained (yield ratio: 64%).

—Synthesis of Intermediate Product (B)—

Under ice cooling, 1.3 ml (0.014 mol) of phosphoryl chloride was carefully added dropwise to 10 ml of N,N-dimethylformamide so as not to generate an excess amount of heat. After the temperature returned to room temperature, the solution was stirred for 1 hour. Next, 4 g (0.012 mol) of Intermediate Product (A) was added, followed by heating and stirring at 70° C. for 4 hours. After allowing the solution to cool to room temperature, the reaction solution was carefully added dropwise to a solution in which 6.2 g of sodium carbonate was dissolved in 65 ml of water, followed by stirring for 1 hour. Ethyl acetate was added, and extraction was performed two times. After being dehydrated with magnesium sulfate, the solution was filtered, and the solvent of the organic layer was removed by distillation under reduced pressure. The residue was purified using silica gel column chromatography (ethyl acetate/n-hexane=1/8). As a result, 3.4 g of Intermediate Product (B) was obtained (yield ratio: 78%).

—Synthesis of Intermediate Product (C)—

15 ml of ethanol was added to 1.6 g (0.012 mol) of 2-amino-1,1,3-tricyano-1-propene and 2.8 g (0.024 mol) of ethyl pyruvate, and the solution was heated to reflux for 1 hour under nitrogen flow conditions. After allowing the solution to cool, 3 g (0.0083 mol) of Intermediate Product (B) was added to the reaction solution and was heated to reflux for 3 hours under nitrogen flow conditions. After allowing the solution to cool to room temperature, deposited crystals were separated by filtration. As a result, 1.9 g of Intermediate Product (C) was obtained (yield ratio: 43%).

—Synthesis of Exemplary Compound (1)—

0.6 g (1.1 mmol) of Intermediate Product (C), 0.45 g (1.7 mmol) of triphenylphosphine, and 0.3 ml (3.3 mmol) of 1-butanol were dissolved in 20 ml of tetrahydrofuran. Under ice cooling, 0.77 ml (1.7 mmol) of diethyl azodicarboxylate (2.2 mol/1 of toluene solution) was added dropwise to the solution under nitrogen flow conditions. After the temperature returned to room temperature, the solution was stirred for 8 hours, and the solvent was removed by distillation under reduced pressure. After washing the obtained solid content with methanol, crystals were separated by filtration. The obtained crystals were recrystallized using a mixed solvent of dichloromethane and methanol. As a result, Exemplary Compound (1) was obtained. The yield amount was 0.5 g, and the yield ratio was 75%. ¹H NMR (CDCl₃) δ8.91 (d, 1H), 7.58 (d, 1H), 7.16 (d, 1H), 6.35 (d, 1H), 6.00 (s, 1H), 4.02 (t, 2H), 3.95 (m, 2H), 3.41 (t, 4H), 1.89 (m, 1H), 1.30 to 1.72 (m, 20H), 0.89 to 1.02 (m, 15H) ppm

### Example 1

### Preparation of Organic Non-Linear Optical Material

A solution in which 1 part by mass of Exemplary Compound (1) and 10 parts by mass of polycarbonate (manufactured by Mitsubishi Gas Chemical Co., Inc.) were dissolved in 89 parts by mass of cyclopentanone (melting point: 130° C.) was applied using a spin coating method to a glass substrate (5 cm×5 cm) on which an ITO layer was provided, and was dried at 120° C. for 1 hour to obtain a thin film A having a thickness of 1.8 μm.

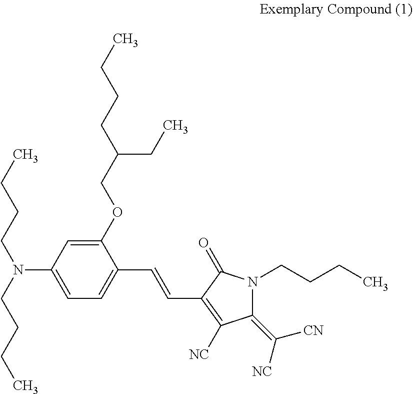 [SMILES: CCCCC(CC)COc1cc(N(CCCC)CCCC)ccc1/C=C/C1=C(C#N)C(=C(C#N)C#N)N(CCCC)C1=O]

### Example 2

An organic non-linear optical material was prepared and evaluated using the same method as in Example 1, except that the following Exemplary Compound (2) was used instead of Exemplary Compound (1) of Example 1.

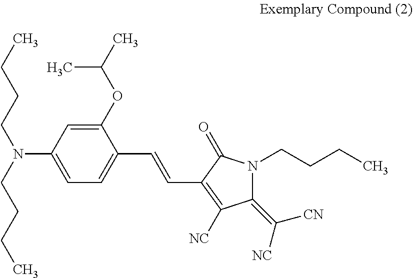 [SMILES: CCCCN1C(=O)C(/C=C/c2ccc(N(CCCC)CCCC)cc2OC(C)C)=C(C#N)C1=C(C#N)C#N]

### Example 3

An organic non-linear optical material was prepared and evaluated using the same method as in Example 1, except that the following Exemplary Compound (8) was used instead of Exemplary Compound (1) of Example 1.

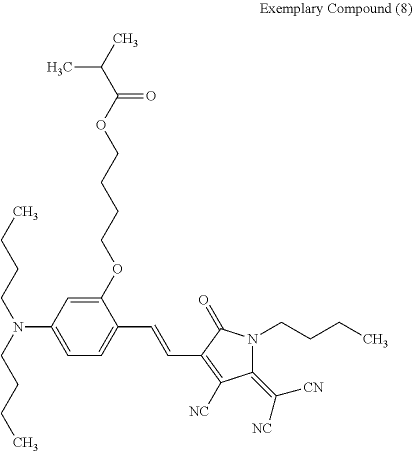 [SMILES: CCCCN1C(=O)C(/C=C/c2ccc(N(CCCC)CCCC)cc2OCCCCOC(=O)C(C)C)=C(C#N)C1=C(C#N)C#N]

### Example 4

An organic non-linear optical material was prepared and evaluated using the same method as in Example 1, except that the following Exemplary Compound (14) was used instead of Exemplary Compound (1) of Example 1.

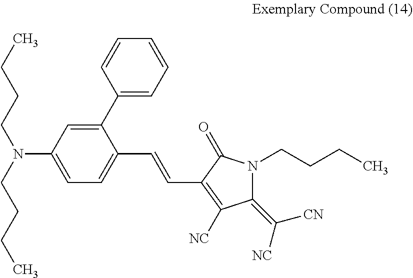 [SMILES: CCCCN1C(=O)C(/C=C/c2ccc(N(CCCC)CCCC)cc2-c2ccccc2)=C(C#N)C1=C(C#N)C#N]

### Example 5

An organic non-linear optical material was prepared and evaluated using the same method as in Example 1, except that the following Exemplary Compound (21) was used instead of Exemplary Compound (1) of Example 1.

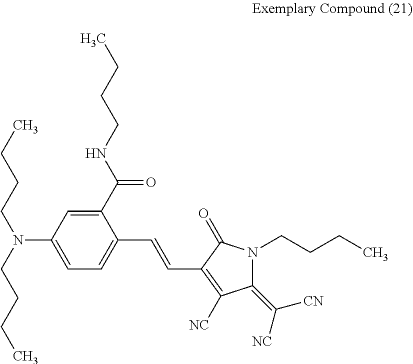 [SMILES: CCCCNC(=O)c1cc(N(CCCC)CCCC)ccc1/C=C/C1=C(C#N)C(=C(C#N)C#N)N(CCCC)C1=O]

### Comparative Example 1

An organic non-linear optical material was prepared using the same method as in Example 1, except that the following Exemplary Compound (X) was used instead of Exemplary Compound (1) of Example 1. However, a non-dissolved product of Exemplary Compound (X) was found during the preparation. Therefore, the dissolution of the non-dissolved product was verified by visual inspection after heating the solution to 40° C. under stirring during the preparation. As a result, a thin film A was obtained.

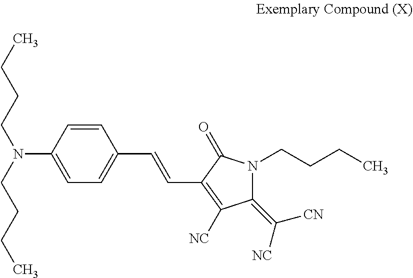 [SMILES: CCCCN1C(=O)C(/C=C/c2ccc(N(CCCC)CCCC)cc2)=C(C#N)C1=C(C#N)C#N]

The above evaluation results are shown in Table 1.

(Evaluation)

—Bleed-Out Evaluation—

During the preparation of the thin film A, the above-described solution was applied using a spin coating method and then was left to stand in an ordinary temperature atmosphere for 30 minutes. At this time, whether or not non-linear optical pigment crystals were deposited on the film surface due to bleed-out was determined by visual inspection. A case where no bleed-out was observed was evaluated as A, a case where bleed-out was observed on a portion of the film surface was evaluated as B, and a case where significant bleed-out was observed on the entire film surface was evaluated as C. In practice, A or B is preferable.

—Electric Field Poling—

The obtained thin film A was provided on a hot plate to perform corona poling on the thin film A. Specifically, in a state where a charging voltage of 17 kV was applied at a distance of 30 mm from the thin film A, the thin film A was held at 140° C. for 0.5 minutes. In the state where the charging voltage was applied, the thin film A was cooled to 40° C., which was lower than the glass transition temperature of the thin film A, for 10 minutes. Next, the charging voltage was removed. Through the above-described process, a thin film B in which a non-linear optical pigment was oriented in the thickness direction was obtained.

—Orientation Efficiency—

In addition, an order parameter was obtained as an index indicating the orientation efficiency of electric field poling.

The order parameter was calculated from the following Expression (1) after measuring absorption spectra of visible ranges of thin films B and C using a visible/infrared polarization spectrophotometer (V-670ST, manufactured by JASCO Corporation):
(1) the thin film B in which the thin film A was poled so as to orient the non-linear optically active compound in the film thickness direction; and
(2) the thin Film C in which the thin film B was held at a temperature, at which poling was performed, for 10 minutes so as to relax the orientation without applying a voltage.

φ=1−Bₜ/A₁ Expression (1)

(In Expression (1), φ represents the order parameter; Bₜ represents the absorbance of the poled thin film B at a wavelength of λmax; and A₁ represents the absorbance of the orientation-relaxed thin film C at the wavelength of λmax.)

The orientation efficiency was evaluated based on three stages: a case where the order parameter was 0.20 or higher was evaluated as A; a case where the order parameter was lower than 0.20 and 0.10 or higher was evaluated as B; and a case where the order parameter was lower than 0.10 was evaluated as C. In practice, A or B is preferable.

—Electro-Optic Constant Evaluation—

In addition, an electro-optic constant (hereinafter, referred to as “r value”) was obtained as an index indicating the non-linear optical performance. r value was calculated from the following Expression (2) after measuring the dependence of the amount of refractive index change of the electric field-poled thin film B on the applied voltage at a wavelength of 1312 nm using a prism coupler (Model: 2010/M, manufactured by Metricon Corporation) including a transparent electrode on a prism surface.

r={(δn/δV)×2×d}}/(n_{TM}³) Expression (2)

(In Expression (2), δn/δV represents the slope of the dependence of the refractive index change on the applied voltage; d represents the thickness (pm) of the thin film B; and n_{TM} represents the refractive index of the thin film B to which a voltage was not applied when a TM wave is incident.)

The electro-optic constant was evaluated based on three stages: a case where the r value was 7.0 or higher was evaluated as A; a case where the r value was lower than 7.0 and 5.0 or higher was evaluated as B; and a case where the r value was lower than 5.0 was evaluated as C. In practice, A or B is preferable.

### [Table 1]

TABLE 1

| | | | Non-Linear | |
|---|---|---|---|---|
| | | | Optical | |
| | | Orientation | Performance | |
| | Bleed-Out | Order | Electro-Optic | |
| | Bleed-Out | Parameter | Constant | Overall |
| | Evaluation | φ | r (pm/V) | Evaluation |
| Example 1 | B | 0.20 A | 7.3 A | A |
| (Exemplary | | | | |
| Compound 1) | | | | |
| Example 2 | B | 0.20 A | 7.1 A | A |
| (Exemplary | | | | |
| Compound 2) | | | | |
| Example 3 | A | 0.22 A | 7.8 A | A |
| (Exemplary | | | | |
| Compound 8) | | | | |
| Example 4 | B | 0.19 B | 6.6 B | B |
| (Exemplary | | | | |
| Compound 14) | | | | |
| Example 5 | B | 0.15 B | 4.8 C | C |
| (Exemplary | | | | |
| Compound 21) | | | | |
| Comparative | C | 0.09 C | 5.3 B | D |
| Example 1 | | | | |
| (Exemplary | | | | |
| Compound X) | | | | |

Overall evaluation was performed based on the evaluation results of the respective items. From the viewpoint of practical use, the evaluation values of the respective items are preferably A or B and more preferably A. Therefore, in the overall evaluation, a case where two or more items were evaluated as A and no items were evaluated as C was evaluated as “A”; a case where one or less items was evaluated as A and no items were evaluated as C was evaluated as “B”; a case where one item was evaluated as C was evaluated as “C”; and a case where two or more items were evaluated as C was evaluated as “D”.

It was found from the above results that, by using the organic compound having non-linear optical activity according to the present invention, bleed-out is suppressed, and the orientation efficiency of electric field poling is significantly improved; as a result, superior non-linear optical performance can be obtained.

In the non-linear optical material according to the present invention, not only non-linear optical performance and but also compatibility with a polymer binder can be simultaneously improved by using a specific organic compound having non-linear optical activity which is superior in non-linear optical performance and the like. In addition, a non-linear optical element including the non-linear optical material according to the present invention can be obtained.

The present invention has been described in detail with reference to the specific embodiment. However, it is obvious to those skilled in the art that various modifications and changes can be made within a range not departing from the scope of the present invention.

The present application is based on Japanese Patent Application (JP2013-099567) filed on May 9, 2013, the entire content of which is incorporated herein by reference.

What is claimed is:

1. An organic non-linear optical material comprising a compound represented by the following Formula (I) and a polymer binder: 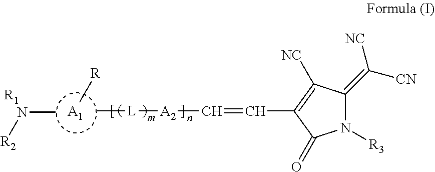
wherein in Formula (I),
R₁ and R₂ each independently represents a substituted or unsubstituted alkyl group or a substituted or unsubstituted aryl group;
R₃ represents a hydrogen atom, a substituted or unsubstituted alkyl group, a substituted or unsubstituted aryl group, a substituted carbonyl group, or a substituted or unsubstituted sulfonyl group;
A₁ and A₂ each independently represents an aromatic group;
L represents —CR₆═CR₇—, —C≡C—, —N═CR₈—, or —CR₉═N—, wherein R₆, R₇, R₈, and R₉ each independently represents a hydrogen atom, a substituted or unsubstituted alkyl group, or a substituted or unsubstituted aryl group;
m represents an integer of 0 or 1;
n represents an integer of 0 to 2, and plural L's, A₂'s and m's may be the same as or different from each other;
R has 3 to 30 carbon atoms and is represented by the following Formula (II); and R may be singular or plural, and plural R's may be the same as or different from each other: 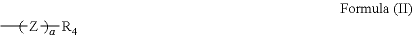
wherein in Formula (II),
Z represents —O—, —S—, —CO—, —SO—, —SO₂—, or —NR₅—, R₅ represents a hydrogen atom, a substituted or unsubstituted alkyl group, or a substituted or unsubstituted aryl group;
R₄ represents a substituted or unsubstituted alkyl group, a substituted or unsubstituted aryl group, or a substituted or unsubstituted heteroaryl group;
a represents an integer of 0 to 3; and plural Z's may be the same as or different from each other.

2. The organic non-linear optical material according to claim 1,
wherein A₁ represents a phenylene group, a naphthylene group, a divalent thiophene ring (thienylene group), a divalent pyrrole ring, or a divalent furan ring.

3. The organic non-linear optical material according to claim 1,
wherein Formula (I) is represented by the following Formula (III): 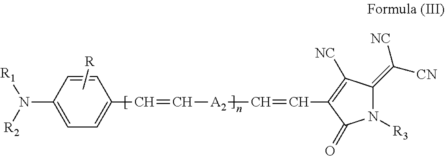
wherein in Formula (III),
R₁ and R₂ each independently represents a substituted or unsubstituted alkyl group or a substituted or unsubstituted aryl group;
R₃ represents a hydrogen atom, a substituted or unsubstituted alkyl group, a substituted or unsubstituted aryl group, a substituted carbonyl group, or a substituted or unsubstituted sulfonyl group;
A₂ represents an aromatic group;
n represents an integer of 0 to 2, and plural A₂'s may be the same as or different from each other;
R has 3 to 30 carbon atoms and is represented by Formula (II); and R may be singular or plural, and plural R's may be the same as or different from each other.

4. The organic non-linear optical material according to claim 1,
wherein in Formula (I), A₂ represents a substituted or unsubstituted phenylene group, a substituted or unsubstituted thienylene group, a substituted or unsubstituted divalent pyrrole ring, or a substituted or unsubstituted divalent thiazole ring.

5. The organic non-linear optical material according to claim 1,
wherein in Formula (I), R has 3 to 30 carbon atoms and represents a substituted or unsubstituted alkyl group, a substituted or unsubstituted alkoxy group, a substituted or unsubstituted aryloxy group, a substituted or unsubstituted alkylthio group, a substituted or unsubstituted arylthio group, or a substituted or unsubstituted acylamino group.

6. An optical element comprising the organic non-linear optical material according to claim 1.

7. An optical modulator comprising the organic non-linear optical material according to claim 1.

8. A compound represented by the following Formula (I): 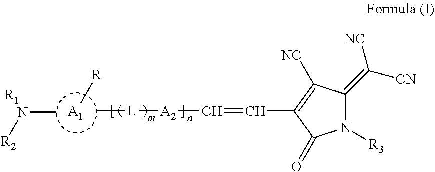
wherein in Formula (I),
R₁ and R₂ each independently represents a substituted or unsubstituted alkyl group or a substituted or unsubstituted aryl group;
R₃ represents a hydrogen atom, a substituted or unsubstituted alkyl group, a substituted or unsubstituted aryl group, a substituted carbonyl group, or a substituted or unsubstituted sulfonyl group;
A₁ and A₂ each independently represents an aromatic group;
L represents —CR₆═CR₇—, —C≡C—, —N═CR₈—, or —CR₉═N—, wherein R₆, R₇, R₈, and R₉ each independently represents a hydrogen atom, a substituted or unsubstituted alkyl group, or a substituted or unsubstituted aryl group;
m represents an integer of 0 or 1;
n represents an integer of 0 to 2, and plural L's, A₂'s and m's may be the same as or different from each other;
R has 3 to 30 carbon atoms and is represented by the following Formula (II); and R may be singular or plural, and plural R's may be the same as or different from each other: 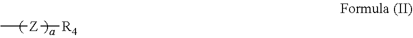
wherein in Formula (II),
Z represents —O—, —S—, —CO—, —SO—, —SO₂—, or —NR₅—, R₅ represents a hydrogen atom, a substituted or unsubstituted alkyl group, or a substituted or unsubstituted aryl group;
R₄ represents a substituted or unsubstituted alkyl group, a substituted or unsubstituted aryl group, or a substituted or unsubstituted heteroaryl group;
a represents an integer of 0 to 3; and plural Z's may be the same as or different from each other.
